(19) United States
(12) Patent Application Publication
(10) Pub. No.: US 2026/0210960 A1
(43) Pub. Date: Jul. 23, 2026

(54) REAGENT MATRIX COMPRISING CELLULOSE NANOFIBRILS AND ITS USE FOR DETECTING ANALYTES

(71) Applicant: Solventum Intellectual Properties Company, Maplewood, MN (US)

(72) Inventors: Deniz YUKSEL YURT, Maplewood, MN (US); Semra COLAK ATAN, Maplewood, MN (US); Kevin T. REDDY, Maplewood, MN (US); Stephen B. ROSCOE, Maplewood, MN (US); Matthew S. STAY, Maplewood, MN (US); Ki-Joo SUNG, Maplewood, MN (US); Katie F. WLASCHIN, Maplewood, MN (US)

(73) Assignee: Solventum Intellectual Properties Company, Maplewood, MN (US)

(21) Appl. No.: 19/136,036

(22) PCT Filed: Nov. 29, 2023
(86) PCT No.: PCT/IB2023/062018

§ 371 Date: Jun. 5, 2025

Related U.S. Application Data
(60) Provisional application No. 63/432,797, filed on Dec. 15, 2022.

(51) Int. Cl. G01N 33/548; G01N 33/543; G01N 33/552

(52) U.S. Cl. CPC G01N 33/548; G01N 33/54306; G01N 33/552

(57) ABSTRACT

Described herein is a reagent matrix comprising (a) a plurality of a first capture component, wherein the first capture component comprises a first analyte capture site: (b) a plurality of a second capture component, wherein tire second capture component comprises a second analyte capture site, and a cellulose binding domain (CBD) or a Carbohydrate binding module (CBM), wherein at least one of the first or second capture components comprises a detection medium; and (c) a plurality of cellulose nanofibrils. In one embodiment, the reagent matrix is used in conjunction with a porous substrate for detection of an analyte.

### TECHNICAL FIELD

Disclosed herein is a reaction matrix comprising at least two different capture components and a plurality of cellulose nanofibrils, which when contacted with an analyte of interest can enable the detection of an analyte.

### SUMMARY

Point of care (POC) devices are a powerful tool for rapid and inexpensive testing in clinical settings as well as the monitoring of food safety and environmental conditions. Most POC devices obviate the need for centralized laboratory testing and provide qualitative or semi-quantitative results without specialized equipment. There has been an increasing demand for POC testing worldwide due to persistent and emerging pandemics like tuberculosis. HIV/AIDS, and COVID-19.

One type of POC device is a lateral flow assay. In typical lateral flow assays, a liquid sample (or its extract) is added to the device and the capillary flow of a liquid is used to move the analyte of interest to other zones of the device for detection. Because capillary flow is used to move the analyte of interest to the detection zone, these assays can require wait times of 10-30 minutes to “read” the test result.

In vertical flow assays the sample is applied to the device and detection occurs at the point of application. Vertical flow assays are known to have a faster detection time than lateral flow assays, but are often more complicated for end-users, including multiple reaction steps (for example at least 2 different reaction reagents are used along with a tagging solution), which must be done in a certain order and numerous wash steps are needed to clear away reagents.

In one aspect, a reagent matrix is disclosed. The reagent matrix comprising:
(a) a plurality of a first capture component, wherein the first capture component comprises a first analyte capture site; (b) a plurality of a second capture component, wherein the second capture component comprises a second analyte capture site, and a cellulose binding domain (CBD) or a carbohydrate binding module (CBM), wherein at least one of the first or second capture components comprises a detection medium; and
(c) a plurality of cellulose nanofibrils.

In another aspect, a kit for performing an assay for detection of an analyte in a sample is disclosed. The kit comprising (i) a reagent matrix comprising: (a) a plurality of a first capture component, wherein the first capture component comprises a first analyte capture site; (b) a plurality of a second capture component, wherein the second capture component comprises a second analyte capture site, and a cellulose binding domain (CBD) or a carbohydrate binding module (CBM), wherein at least one of the first or second capture components comprises a detection medium; and (c) a plurality of cellulose nanofibrils; and (ii) a porous substrate.

In yet another aspect, a method for detecting the presence or amount of an analyte in a sample is disclosed. The method comprising: ii) combining the sample; a reagent matrix comprising: (a) a plurality of a first capture component, wherein the first capture component comprises a first analyte capture site; (b) a plurality of a second capture component, wherein the second capture component comprises a second analyte capture site, and a cellulose binding domain (CBD) or a carbohydrate binding module (CBM), wherein at least one of the first or second capture components comprises a detection medium; and (c) a plurality of cellulose nanofibrils; and a carrier solution in a vessel to form a testing sample; (ii) contacting the testing sample to a porous substrate; and (iii) detecting a signal produced by an aggregation of the analyte with the reagent matrix on a surface of the porous substrate.

The above summary is not intended to describe each embodiment. The details of one or more embodiments of the invention are also set forth in the description below. Other features, objects, and advantages will be apparent from the description and from the claims.

### DETAILED DESCRIPTION

As used herein, the term
“a”. “an”, and “the” are used interchangeably and mean one or more; and
“and/or” is used to indicate one or both stated cases may occur, for example A and/or B includes, (A and B) and (A or B).

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

As used herein, “comprises at least one of” A, B. and C refers to element A by itself, element B by itself, element C by itself, A and B, A and C, B and C, and a combination of all three.

In the present disclosure, a novel technique for detection of a particular analyte is disclosed.

In an aggregation-type assay, the target analyte binds to particular capture agents to form a detectable agglomerate. In the present disclosure, an aggregation-type assay is performed using a plurality of cellulose nanofibrils in addition to capture components to improve the detection for a particular analyte. The method of the present disclosure is a simplified process for a vertical flow type assay, wherein a single step can be used to lyse the sample, and capture and label the target analyte. The method is simple, fast, and can provide detection limits comparable or better than commercially available tests.

In the method of the present application, the target analyte is used to complex two different capture components, wherein at least one of the capture components can also bind to a cellulose nanofibrils. This agglomeration can occur in situ and is then applied to a porous substrate, which captures the agglomerate on the substrate surface.

### Reagent Matrix

The reagent matrix of the present disclosure comprises at least two different components that can capture the target analyte. The analyte (or target analyte) is the compound or composition of interest to be detected. The analyte can be biologically-derived or present in a biologically-derived testing sample fluid. Examples of such analytes may include therapeutic drugs, drugs of abuse, pharmaceutical metabolites, hormones, peptides, polypeptides, proteins including immunoglobulins, polysaccharides, nucleic acids, and combinations thereof. In another embodiment, the analyte may be an agent of environmental interest, such as a pest control product, an environmental toxin, a halogenated substance, or dioxins and furans. In another embodiment, the analyte may be an agent of food safety interest, such as pathogens (e.g., bacteria, virus, fungi, etc.), allergens, pesticides, genetically modified organisms, and toxins.

The first capture component comprises a first site for capture of the analyte. The first capture site is capable of recognizing a particular spatial and/or chemical structure of the analyte.

The second capture component comprises a second capture site which also is capable of recognizing a particular spatial and/or chemical structure of the analyte. The second capture component also includes a cellulose binding domain (CBD) and/or a carbohydrate binding module (CBM).

In one embodiment, the capture site may be an antibody. As used herein, the term antibody covers not only antibodies, but any polypeptide or protein having a binding domain which is, or is homologous to, an antibody binding domain. These can be derived from natural sources, or they may be partly or wholly synthetically produced. Examples of antibodies are the immunoglobulin isotypes and their isotypic subclasses; fragments which comprise an antigen binding domain such as Fab, scFv, Fv, dAb, Fd; and diabodies. Antibodies useful in the present disclosure include those specifically reactive with the target analyte. Antibody capture sites are preferable when testing of biological samples. Such antibodies are preferably IgG or IgM antibodies or mixtures thereof, which are essentially free of association with antibodies capable of binding with non-analyte molecules. The antibodies may be polyclonal or monoclonal and are commercially available or may be obtained by mouse ascites, tissue culture or other techniques known to the art. The use of mixtures of monoclonal antibodies of differing antigenic specificities or of monoclonal antibodies and polyclonal antibodies may be desired. A typical description of hybridoma procedure for the production of monoclonal antibodies may be found for example in Wands, et al., Gastroenterology 80 pages 225-232(1981); and U.S. Pat. No. 4,515,893 issued to Kung, et al. It is further contemplated that fragments of antibody molecules may be used as specific binding reagents according to the present disclosure including half antibody molecules and Fab, Fab′ or F(ab′)2 fragments known in the art.

In another embodiment, the capture site is a capture protein such as an engineered protein, peptide aptamers, or affimer proteins. A plethora of protein engineering methods are known in the art to generate peptides and proteins with enhanced or novel functions. One such methodology is directed evolution, which involves creation of a random library displaying a broad set of protein variants. The library is then displayed using one of the various technologies available e.g., phage, ribosome, mRNA, or cell surface display. Following selections, affinity maturation is obtained by adding diversity back to the library by DNA modification (e.g., error prone PCR DNA shuffling, etc.). The cyclic selection process is repeated until the desired binders are produced. In addition to molecular biology-based approaches, chemical combinatorial methods such as DNA-encoded libraries and one-bead-one-compound libraries can be used to create high affinity binding peptides. Other approaches to obtain new binding domains also include semi-rational design and computational techniques. An overview of the techniques mentioned here can be found in Banta et al. in Annu Rev. Biomed Eng., vol. 15, pages 93-113 (2013); and Zhao et al, in Expert Opin. Drug Discov., vol. 14, pages 735-753 (2019).

In another embodiment, the capture site is an aptamer, such as DNA, RNA, or peptide aptamers. RNA and DNA aptamers bind to their targets with high selectivity and sensitivity due to their structural confirmation. The aptamers can be produced by techniques known in the art such as SELEX (systematic evolution of ligands by exponential enrichment). See Ellington et al. in Nature, v. 346, pages 818-822 (1990), and Tuerk, et al. in Science, v 249, pages 505-510 (1990). Peptide aptamers are alternative binding molecules where a 5-20 residue peptide is typically grafted onto a neutral scaffold which undergoes a selection procedure. Peptide aptamers can be produced and selected using display strategies known in the art. See Reverdatto et al. in Curr. Top. Med. Chem. v. 15, iss. 12, pages 1082-1101 (2015).

The second capture component further comprises a cellulose binding domain (CBD) and/or a carbohydrate binding module (CBM) which can bind to the cellulose nanofibrils. CBD and CBM are sites that have an affinity toward cellulosic materials. CBD and CBM sequences and how to obtain these groups are known in the art. See for example, U.S. Pat. Publ. No. 2019/0113512 (Sikes Johnson et al) and U.S. Pat. No. 5,496,934 (Shoseyov et al.). In some embodiments, the CBM is CBM1, CBM2, CBM3, CBM4, CBM5, CBM6, CBM9, CBM10, CBM11, CBM12, CBM14, CBM15, CBM17, CBM18. CBM19, CBM20, CBM21, CBM25, CBM27, CBM28, CBM32, CBM33, CBM48, or CBM49. The nucleic acid and amino acid sequences of CBMs contemplated herein have been described, such as those disclosed in www.carypedia.org/index.php/Carbohydrate-binding modules, and can be readily identified by one of ordinary skill in the art using a Basic Local Alignment Search Tool (BLAST). Orthologs of CBDs have been described in various species, including, but not limited to Micromonospora mirobrigensis (GenBank ID: SCF42127.1). Mycobacterium tuberculosis (GenBank ID: CNEINW97.1), Micromonospora nigra (GenBank ID: SCL15442.1). Micromonospora mirobrigensis (GenBank ID: SCF04121.1). Cellulomonas Fimi (PDB: IEXH_A), Mycobacterium kansasii 732 (GenBank: EUA13076.j), Ruminococcus albus 8 (GenBank: EGC02462.1), Leifsonia aquatic (NCBI Reference Sequence: WP_021763186.1). Schizosaccharomyces pombe (NCB1 Reference Sequence: NP_593986.1), Desulfitobacterium hafniense (GenBank: CDX04743.1). CBDs expressed in other species that are known to one of ordinary skill in the art, such as CBDs of families I, II, III and IV disclosed, for instance, in Tomme et al., J Chromatogr B Biomed Sci Appl (1998) 715(0):283-96, are also contemplated herein.

The second capture component may be a bifunctional fusion protein as disclosed in U.S. Pat. Publ. No. 2019/0113512 (Sikes Johnson et al.) herein incorporated by reference.

The first capture component, the second capture component, or both comprise a detection medium. The detection medium may be any molecule or particle bound or conjugated to the capture site, which can enable detection. The signal may be one that is detected visually (for example by eye) or one that is detected with an instrument. The detection medium may be a colorant, a photoluminescent substance, a chemiluminescent substance, a radio-label, a magnetic material, or combinations thereof.

The first and second capture sites are selected to have a specific binding affinity for different portions of the analyte, thereby sandwiching the analyte therebetween. The capture sites may be naturally derived or synthetically produced. The first capture site specifically binds to and is therefore complementary to a particular spatial and/or chemical structure of the analyte, while the second capture site specifically binds to and is therefore complementary to a particular spatial and/or chemical structure of another portion the analyte. It is known in the art how to select such sandwich pairs. See for example, a review article by Mirica et al. in From. Bioeng. Biotechnol. vol. 10, article 922772, June 2022.

In one embodiment, the capture component comprises a particle, wherein the capture site is bound or conjugated on or to the particle via passive adsorption or covalent attachment as known in the art. Typically, these particles may comprise a synthetic polymer (such as a latex), glass, metal, metal oxide, liposomes, pollen spores, red blood cells, carbohydrates (such as dextans, agarose, or cellulose), microoganisms including viruses, and combinations thereof. In one embodiment, the particles comprise cellulose or latex. Latexes are commercially available and the polymer particles therein may be derived from acrolein, acrylate, methyl acrylate, methacrylate, methyl methacrylate, glycidyl methacrylate, styrene, vinyl toluene, and t-butyl styrene monomers and mixtures thereof. In some embodiments, the polymer particles of the latex may optionally containing crosslinking agents such as divinyl benzene and butadiene. Techniques for preparing such latexes are well-known as are surface modifications used to attach binding pair members to the particle surfaces. Exemplary U.S. patents that describe either latex particles, capture sites that can be attached to the particles, and/or coupling methods for attaching the capture sites to the particle surfaces include U.S. Pat. No. 4,064,088 (Renner); U.S. Pat. No. 4,210,723 (Dorman, et al.); U.S. Pat. No. 4,264,766 (Fisher): U.S. Pat. No. 3,857,931 (Hager); U.S. Pat. No. 4,253,844 (Limet et al.); and U.S. Pat. No. 4,397,960 (Moussebois et al.), each herein incorporated by reference. Cellulose particles can be made from cellulose using various techniques, such as pulverized cellulose, which may have more bar-like of fibrous shapes as disclosed in U.S. Pat. No. 5,123,962 (Komuro et al.); or dissolving cellulose and then processing it through high shear or stirring as disclosed in JP H 0576496 (Shigeru et al.), coagulating as disclosed in U.S. Pat. No. 6,225,461 (Akimoto et al.), or drying the dispersion as disclosed in U.S. Pat. No. 8,629,187 (Shimomi et al.), each herein incorporated by reference. A commercially available cellulose includes nanocellulose available for example by Asahi Kasei under the trade designation “NANOAC”.

In one embodiment, a dye is added to the particle, such that when a substantial amount of particles agglomerate at the surface of the porous substrate, a signal can be visually observed.

The size of the particles and the pores/porosity of the porous substrate are selected such that unbound particles can pass through the porous substrate, while when the analyte of interest is sandwiched between the first and second capture components and the sandwiched analyte does not pass through the porous substrate. In one embodiment, the particles have an average diameter of at least 0.1, 0.3, 0.5, or even 1 micrometer and at most 10, 7.5, 5.2, or even 1.5 micrometers. The particle size may be determined using techniques known in the art such as scanning electron microscope or light scattering detection.

In addition to the first capture component and the second capture component, the reagent matrix further comprises a plurality of cellulose nanofibrils. Cellulose nanofibrils are a particular type of cellulose material. As used herein cellulose nanofibrils includes fibrillated cellulose (both nanofibrillated cellulose and microfibrillated cellulose).

Cellulose nanofibrils can be produced from cellulosic materials, such as wood pulp, bacteria, cellulose-containing sea animals (e.g., tunicate), or cotton, with wood pulp being the most commonly used.

The cellulose nanofibrils can be made using mechanical treatments, such as high-pressure homogenization, high-energy ball mills, microfluidizers, ultra-low crushing, and other such method; enzymatic treatments; and/or chemical treatments such as strong acid hydrolysis, oxidation, chemical functionalization, or combinations thereof.

Cellulose nanofibrils are constituted of cellulose, a linear polymer of beta (I to 4) linked D-glucose units, the chains of which arrange themselves to form crystalline and amorphous domains.

The physical dimensions of cellulose nanofibrils can vary depending on the raw material and how it was treated. Typically, the microfibrillated cellulose comprises long thin fibers with a large size distribution, including individual fibers with a nanometer diameter, but there are a lot of bigger fibers with the fibers forming a network structure. Nanofibrillated cellulose tends to comprise individual fibrils, with nanoscale diameters and a narrow size distribution. In one embodiment, the cellulose nanofibrils have an average cross-sectional distance (longest dimension of a cross-section of the cellulose material perpendicular to the length) of at least 2, 4, or even 5 nanometers (nm) and at most 10, 20, 30, or even 50 run; and an average length (longest dimension of the cellulose nanocrystal) of at least 50, 75, or even 100 nm and at most 150, 200, 250, 500, 750, or even 1000 nm. In another embodiment, the cellulose nanofibrils have an average cross-sectional distance (longest dimension of a cross-section of the cellulose material, perpendicular to the length) of at least 100, 500, 1000, even 2000 nanometers (nm) and at most 1, 2, 5, 10, 50, 75, or even 100 micrometer; and an average length (longest dimension of the cellulose nanocrystal) of at least 0.5, 1, 2, 5, 10, 50, or even 100 micrometers and at most 150, 200, 250, 500, 750, or even 1000 micrometer. The cross-sectional morphology of the nanofibrils is typically square, but can be rectangular, or rounded. Typically, the cellulose nanofibrils have a high aspect ratio (ratio of height versus length). In one embodiment, the cellulose nanofibrils have an aspect ratio of 10 to 200, or even 100-150. The dimensions of the cellulose nanofibrils may be determined based on transmission electron microscopy (TEM), scanning electron microscopy (SEM), atomic force microscopy, or by other suitable means. Typically, the morphology is determined on dried samples. In one embodiment, the cellulose nanofibrils have an average surface area of at least 30, 40, 50, 70, or even 100 m/g. In one embodiment, the cellulose nanofibrils have an average surface area of at most 100, 150, 200, 300, 400, or even 500 m²/g.

The zeta potential measures the potential difference existing between the surface of a solid particle immersed in a conducting liquid (e.g., water) and the bulk of the liquid of the cellulose nanofibril surface. The cellulose nanofibrils have a zeta potential higher (i.e., less negative) than −50, −45, −40, −35, −30, or even −25 mV based on dynamic light scattering.

The cellulose nanofibrils typically have a pH of less than 7.5, 7.0, 6.5, or even 6.0 and greater than 4.5, 5.0, or even 5.5 when measured at ambient conditions.

Cellulose nanofibrils may be obtained, for example, from CelluForce, Montreal, Canada; Melodea Ltd., Israel: American Process Inc. Atlanta. GA; Blue Goose Biorefineries Inc., Saskatoon, Canada the USDA Forest Products Laboratory, Madison, WI via the University of Maine; and Weidmann Fiber Technology, Rapperswil, Switzerland.

In one embodiment disclosure, the cellulose nanofibrils are present in a ratio of at least 0.5:1; 0.75:1; 1:1; or even 1:2 capture component to cellulose nanofibrils. In one embodiment disclosure, the cellulose nanofibrils are present in a ratio of at most 1:1; 1:2; 1:4; 1:5; 1:8; 1:10; 1:15; or even 1:20 capture component to cellulose nanofibrils.

Advantageously, the reagent mixture can be substantially free liquid (in other words comprising less than 5, 3, 2, 1, 0.5, or even 0.1% by weight of a liquid, such as water, alcohol, etc. or even no liquid is detectable). The reagent mixture may be lyophilized, or similarly dried. The reagent mixture being substantially free of water can enable improved shelf-life.

In one embodiment, the reagent matrix further comprises a wetting or lysing agent, such as a solvent (e.g., alcohol) or a surfactant.

### Porous Substrate

In one embodiment, a test sample is applied to a porous substrate. Although not wanting to be bound by theory, it is believed that capture components interact with the target analyte and the plurality of nanofibrils and, upon application to the porous substrate, the aggregated product is concentrated at the surface of the porous substrate while the unbound reagents are drawn away from the surface.

The porous substrate of the present disclosure is a layer comprising a series of interconnected pores from a first major surface of the porous substrate to an opposing second major surface of the porous substrate.

The porous substrate is an organic material, preferably a polymeric material, which can be a porous film or nonwoven.

In one embodiment, the porous substrate is a nonwoven web, which may include nonwoven webs manufactured by any of the commonly known processes for producing nonwoven webs. As used herein, the term “nonwoven web” refers to a fabric that has a structure of individual fibers or filaments which are randomly and/or unidirectionally interlaid in a mat-like fashion. For example, the fibrous nonwoven web can be made by carded, air laid, spunlaced, spunbonding or meltblowing techniques or combinations thereof, Spunbonded fibers are typically small-diameter fibers that are formed by extruding molten thermoplastic polymer as filaments from a plurality of fine, usually circular capillaries of a spinneret with the diameter of the extruded fibers being rapidly reduced. Meltblown fibers are typically formed by extruding the molten thermoplastic material through a plurality of fine, usually circular, die capillaries as molten threads or filaments into a high velocity, usually heated gas (e.g., air) stream, which attenuates the filaments of molten thermoplastic material to reduce their diameter. Thereafter, the meltblown fibers are carried by the high velocity gas stream and are deposited on a collecting surface to form a web of randomly disbursed meltblown fibers. Any of the non-woven webs may be made from a single type of fiber or two or more fibers that differ in the type of thermoplastic polymer, thickness thereof, or both. Further details of manufacturing methods of useful nonwoven webs have been described by Wente in Indus. Eng. Chem., v. 48, pages 1342-1346 (1956). In one embodiment, the microfibers have an effective fiber diameter of at least 0.5 and at most 16 micrometers.

In some embodiments, the porous substrate is a porous membrane such as a thermally-induced phase separation (TIPS) membrane. TIPS membranes are often prepared by forming a homogenous solution of a thermoplastic material and a diluent, and optionally including a nucleating agent, by mixing at elevated temperatures in plastic compounding equipment, e.g., an extruder. The solution can be shaped by passing through an orifice plate or extrusion die, and upon cooling, the thermoplastic material crystallizes and phase separates from the diluent. The crystallized thermoplastic material is often stretched. The diluent is optionally removed either before or after stretching, leaving a porous polymeric structure. Porous membranes are further disclosed in U.S. Pat. No. 4,539,256 (Shipman), U.S. Pat. No. 4,726,989 (Mrozinski), U.S. Pat. No. 4,867,881 (Kinzer), U.S. Pat. No. 5,120,594 (Mrozinski), U.S. Pat. No. 5,260,360 (Mrozinski et al.), U.S. Pat. No. 5,962,544 (Waller), and U.S. Pat. No. 6,096,293 (Mrozinski et al.) all of which are assigned to 3M Company (St Paul, MN), and each incorporated herein by reference.

In some embodiments, the substrate is a porous membrane such as a solvent-induced phase separation (SIPS) membrane. SIPS membranes are often made by preparing a homogeneous solution of a polymer in first solvent(s), casting the solution into desired shape, e.g., flat sheet or hollow fiber, contacting the cast solution with another second solvent that is a non-solvent for the polymer, but a solvent for the first solvent (i.e., the first solvent is miscible with the second solvent, but the polymer is not). Phase separation is induced by diffusion of the second solvent into the cast polymer solution and diffusion of the first solvent out of the polymer solution and into the second solvent, thus precipitating the polymer. The polymer-lean phase is removed and the polymer is dried to yield the porous structure. SIPS is also called Phase Inversion, or Diffusion-induced Phase Separation, or Nonsolvent-induced Phase Separation, such techniques are commonly known in the art.

Useful porous substrates include symmetric, asymmetric, or multizone membranes, as well as multiple layers of such membranes. A symmetric membrane is one having substantially the same average pore size and/or porosity throughout its thickness. An asymmetric membrane is a membrane having a linear or non-linear gradient in average pore size and/or porosity extending from one major surface to an opposing major surface of the fluoroplastic substrate. In other words, the ratio of the average pore size of the one major surface with the larger pores to the average pore size of the opposing surface is greater than 3 or even greater than 4. A multizone membrane is a membrane having two or more substantially distinct through-thickness zones, or layers having different average pore sizes and/or different porosities. Multizone membranes are often designated by the number of layers or zones, (e.g., a 2-zone membrane has two substantially distinct zones having different average pore sizes or different porosities).

The porous substrate is an organic material, preferably a polymeric material. In one embodiment, the porous substrate may be formed from any suitable polymeric material. Suitable polymeric materials include polyolefins, poly(isoprenes), poly(butadienes), fluorinated polymers, chlorinated polymers, polyamides, polyimides, polyethers, poly(ether sulfones), poly(sulfones), poly(vinyl acetates), polyesters such as poly(lactic acid), copolymers of vinyl acetate such as poly(ethylene)-co-poly(vinyl alcohol), poly(phosphazenes), poly(vinyl esters), poly(vinyl ethers), poly(vinyl alcohols), poly(carbonates), fiberglass, and the like, and combinations thereof.

Suitable polyolefins include poly(ethylene), poly(propylene), poly(1-butene), copolymers of ethylene and propylene, alpha olefin copolymers (such as copolymers of ethylene or propylene with 1-butene, 1-hexene, 1-octene, and 1-decene), poly(ethylene-co-1-butene), poly(ethylene-co−1-butene-co−1-hexene), and the like, and combinations thereof.

Suitable fluorinated polymers include poly(vinyl fluoride), poly(vinylidene fluoride), copolymers of vinylidene fluoride (such as poly(vinylidene fluoride-co-hexafluoropropylene)), copolymers of chlorotrifluoroethylene (such as poly(ethylene-co-chlorotrifluoroethylene)), and the like, and combinations thereof.

Suitable polyamides include poly(iminoadipolyliminohexamethylene), poly(iminoadipolyliminodecamethylene), polycaprolactam, and the like, and combinations thereof. Suitable polyimides include poly(pyromellitimide), and the like, and combinations thereof.

Suitable poly(ether sulfones) include poly(diphenylether sulfone), poly(diphenylsulfone-co-diphenylene oxide sulfone), and the like, and combinations thereof.

Suitable copolymers of vinyl acetate include poly(ethylene-co-vinyl acetate), such copolymers in which at least some of the acetate groups have been hydrolyzed to afford various poly(vinyl alcohols), and the like, and combinations thereof.

Typically, the porous substrate is free or substantially free of cellulose and groups that would specifically bind to the first and/or second binding component as it can lead to non-specific binding of the reagents to the porous substrate, causing background noise.

Typically, the porous substrates made of the polymers described above are used in their unfunctionalized state. However, the porous substrate may be functionalized, for example, surface functionalized, with various groups such as acid groups, and/or neutral hydrophilic moieties with a —OCH₂CH₂— group, etc. to enhance signal especially for small aggregates. Exemplary surface functionalized porous membrane and monomers used to make them are described in U.S. Pat. No. 9,958,364 (Rasmussen et al.) and U.S. Pat. Publ. No. 2021/0031152 (Vail et al.), herein incorporated by reference.

In one exemplary embodiment, the porous substrate has an average pore size that is greater than 200, 500, 750, 1000, 2000, 3000, or even 5000 nanometers (nm). In one exemplary embodiment, the porous substrate has an average pore size that is less than about 100, 50, 25, 20, 10, 5, 3, or even 2 μm (micrometers). The average pore size of the porous substrate can be optimized for the particular capture components used and the target analyte. The average pore size can be measured using techniques known in the art, for example, optical microscopy, computed tomography (CT) scanning, or liquid porosimetry.

In some biomaterial assays, proteins are immobilized onto the surface of substrates to detect analytes. In the present application, no immobilized proteins are bound to the surface of the substrate prior to contact with the reagent matrix. In one embodiment, the porous substrate is substantially free (i.e., comprises less than 0.5% by weight, or even no detectable amount) of immobilized proteins prior to contact with the reagent matrix.

In one embodiment, the porous substrate is disposed on an absorbent layer. The absorbent layer or substrate pad, typically located below the porous substrate, opposing where the sample mixture is dispensed, draws the sample, through to the absorbent material below. The absorbent layer can be generated from any material capable of wicking fluid by way of capillary action, such as paper, cellulose, and cellulose derivatives such as cellulose acetate and nitrocellulose, fiberglass, cloth, cotton, polyester, polyolefin such as polyethylene, films of polyvinyl chloride, and the like. In one embodiment, the absorbent layer comprises a dried gel such as silica gel, agarose, dextran, or gelatin.

The selection of material for the absorbent layer is not critical and a variety of fibrous filter materials can be used, including one or more layers of the same or different materials, providing that the material selected is compatible with the target analyte and the assay reagents. Any conventionally employed absorbent material that is capable of drawing or wicking fluid through a porous membrane, such as for example, by capillary action, can be used in the present invention. The absorbent material should be capable of absorbing a volume of fluid test sample that is equivalent or greater than the total volume capacity of the material itself. Useful known materials include cotton, cotton linter, cellulose acetate fibers, polyester, polyolefin, or other such materials. The absorbent material provides a means to collect the sample by providing uniform “suction” to deliver the sample from the well, through the reaction zone, and down into the absorbent material. Thus, the absorbent body also acts as a reservoir to hold the sample, and various reagents that are used when the assay is performed. Accordingly, when used in assays where relatively large volumes of fluid are used, the absorbent material should have high absorbent capacity so as to prevent or minimize the possibility of back-flow of sample and reagents from the absorbent body back into the reaction membrane.

The absorbent layer is placed on the opposite side of the porous substrate from where the sample matrix is added. In one embodiment, the absorbent layer is fixedly attached to the porous substrate, for example, by using adhesive.

### Method

In one embodiment, the multilayered substrate of the present disclosure is used in a downward or vertical flow assay, wherein the multilayered substrate is placed in a testing device comprising a test area. In the test area, the porous substrate faces outward with the absorbent layer there beneath and in vertical communication with the porous substrate.

Exemplary biological samples that can be sampled include: body fluids and tissue samples. Body fluids can include, amniotic fluid, aqueous humor, vitreous humor, bile, blood, cerebrospinal fluid, chyle, endolymph, perilymph, female ejaculate, lymph, mucus (including nasal drainage and phlegm), pericardial fluid, peritoneal fluid, pleural fluid, pus, rheum, saliva, sputum, synovial fluid, vaginal secretion, semen, blood, serum, or plasma. Tissue samples can include organ or tissue extract such as that taken from placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, testis, skin, blood or buffy coat sample of blood. Additional examples of organs and tissues from any biological source are well known to a person of ordinary skill in the art and such embodiments are within the purview of the methods provided herein. In one example, swabbing from a nasal cavity, throat, or other body pan is used for point of care testing.

In environmental testing and food safety testing, the test sample, may or may not be a liquid. For example, a water sample or liquid food sample may be the sample, or a swab may be used to wipe the surface of a food preparation surface, container, or high traffic area.

The reagent matrix is dispersed in liquid, if already not done so, and the sample is added to the reagent matrix to form a test sample. In one embodiment, the reagent matrix is in a dried form and a liquid, such as a buffer and/or surfactant can be used to disperse the capture component reagents and cellulose nanofibrils. Buffers can include those known in the art, especially biological buffers. A surfactant can be used to aid in the denaturation of the analyte and/or sample. Such surfactant includes those known in the art. Suitable buffer solutions to be used in this disclosure include but are not limited to acetate buffer, buffered saline, citrate buffer, barbital buffer, borate buffer, phosphate buffer, or buffer prepared with tris(hydroxyl-methyl)aminomethane (TRIS), N-(2-acetamido)iminodiacetic acid (ADA), piperazine-1,4-bis(2-ethanesulfonic acid) (PIPES), N-(2-acetamido)-2-aminoethanesulfonic acid (ACES), N,N-bis(2-hydoxyethyl)-2-aminoethanesulfonic acid (BES), 3-morpholinopropanesulfonic acid (MOPS), N-tris(hydroxymethyl)methyl-2-aminoethanesulfonic acid (TES), 2,4-(2-hydroxy ethyl)−1-piper azinyl ethanesulfonic acid (HEPES), 3,4-(2-hydroxyethyl)-1-piperazinyl propanesulfonic acid (EPPS), N-tris(hydroxymethyl)methylglycine (Tricine), N,N-bis(2-hydroxyethylglycine (Bicine), N-cyclohexyl-2-aminoethanesulfonic acid (CHES), N-cyclohexyl-3-aminopropanesulfonic acid (CAPS), and/or buffer containing one or more than one buffer salts selected from KH₂PO₄, KH₂PO₄, Na₂HPO₄, NaH₂PO₄, NaHCOs, NaBO₄, (NH4)₂CO₃. In one embodiment, the buffer is a multifunctional buffer as disclosed in U.S. Pat. No. 7,531,362 (Chan, herein incorporated by reference), which comprises (1) a biological buffer to maintain the pH between about 7.0 to 10.0; (2) at least one surfactant to reduce non-specific binding of assay reagents while simultaneously avoiding inhibition of a specific binding interaction: (3) a high molecular weight polymer as a dispersing and suspending reagent having a molecular weight in a range of from about 2×10² to about 2×10⁶D; (4) a pH stabilizer to maintain the pH of the multifunctional buffer between about pH 7.0 to 10.0; (5) an ionic salt to reduce the non-specific binding of antibodies; (6) at least one preservative to reduce bacterial and microbial growth; and (7) a calcium chelator to prevent a whole blood test sample from clotting.

The test sample including the sample and the reagent matrix in a liquid medium and optional surfactant is mixed to enable agglomeration of the analyte with the first and second capture components and the cellulose nanofibrils. The test sample is then disposed onto the test area of the testing device. Although not wanting to be limited by theory, it is believed that the agglomerated sample is captured on the surface of the porous substrate layer, while the reagents flow vertically or downwardly. After application of the test sample, it may be advantageous to include one or more washing steps, for example by adding a wash solution such as a buffer dropwise onto the testing area. It is believed that the capturing of the analyte and concentrating it on the surface of the porous substrate enables good detection of the analyte. Visual detection (in other words, by a human eye) or with an instrument (such as a spectrophotometer, a fluorescence detector, Geiger counter, etc.) may be used to determine if the analyte is present and/or the amount of analyte present in the sample.

In one embodiment, the testing device is one such that multiple analytes (for example 2, 3 or even more different analytes) are simultaneously detected. For example, a first capture component with a detection medium 1 may be used to detect analyte 1, while another first capture component with a detection medium 2 may be used to detect analyte 2. In one embodiment, detection medium 1 and 2 are both colorimetric and the observed color is additive. In another embodiment, detection medium 1 and 2 use different detection mechanisms (e.g., colorimetric and radio-labeled).

### EXAMPLES

Unless otherwise noted, all parts, percentages, ratios, etc. in the examples and the rest of the specification are by weight, and all reagents used in the examples were obtained, or are available, from general chemical suppliers such as, for example. Sigma-Aldrich Company, Saint Louis, Missouri, or may be synthesized by conventional methods.

### Materials and Methods

4-Morpholinoethanesulfonic acid (MES) was obtained from Sigma-Aldrich (St. Louis, MO).

(1-Ethyl-3-(3-dimethylaminopropyl)carbodiimide hydrochloride) (EDC), BLOCKER casein in PBS (phosphate-buffered saline, 1% weight to volume), sucrose, TRITON X-100 surfactant, tris(2-carboxyethyl)phosphine hydrochloride (TCEP), and N-hydroxysulfosuccinimide (sulfo-NHS) were obtained from Thermo Fisher Scientific (Waltham, MA).

Boric acid was obtained from VWR International (Radnor, PA).

Antigen 1: SARS-CoV-2 Nucleoprotein (N-Protein) was obtained from Professor Hadley Sikes. Massachusetts Institute of Technology, Cambridge, MA. The preparation of SARS-CoV-2 Nucleoprotein is described in Kim, S., et. al. “Developing a SARS-CoV-2 Antigen Test Using Engineered Affinity Proteins”, Applied Materials Interfaces 2021, 13, 38990-39002 and Supporting Information S1-S23.

Capture Component 1: The SARS CoV-2 antigen binding protein-cellulose binding domain capture affinity reagent (abbreviation: SsoNP.E2-CBD) was prepared as described in Kim, S., et. al. Applied Materials & Interfaces 2021, 13, 38990-39002 and Supporting Information S1-S23. A 60 micromolar stock solution of SsoNP.E2-CBD was prepared in IX PBS (pH 7.4) that contained 2% weight to volume trehalose.

Capture Component 2: See preparation below

Capture Component 3: For Comparative Example C (CE-C), the CBD sequence of SsoNP.E2-CBD was replaced with a cysteine (Cys) group to provide the non-CBD binding mutant SsoNP.E2-Cys. The SsoNP.E2-Cys can be prepared according to general protocols described in Kim. S., et. al. Applied Materials & Interfaces 2021, 13, 38990-39002 and Supporting information S1-S23. A 60 micromolar stock solution of SsoNP.E2-Cys was prepared in IX PBS pH 7.4 that contained 10 mM tris(2-carboxyethyl)phosphine hydrochloride (TCEP).

Microfibrillated cellulose (MFC) was obtained under the trade designation “CELOVA M250R-G” from Weidmann Fiber Technology (Rapperswil. Switzerland) as a white gel with specifications of 2.9% MFC content, pH 8, specific surface area of 234 m²/g, and particle length Da of 9 micrometers reported by the manufacturer. A 0.3% by volume stock solution of MFC was prepared in TX PBS (pH 7.4) that contained 1% by volume TRITON X-100 surfactant.

AVICEL PH-101 microcrystalline cellulose (50 micrometer particle size) was obtained from Sigma-Aldrich (catalog #11363). A 0.3% (weight to volume) stock solution of the AVICEL PH-101 microcrystalline cellulose was prepared in 1×PBS (pH 7.4) that contained 1% by volume TRITON X-100 surfactant.

Sigmacell cellulose powder (20 micrometer particle size) was obtained from Sigma-Aldrich (catalog #S3504). A 0.3% weight to volume stock solution of the Sigmacell cellulose was prepared in 1×PBS (pH 7.4) that contained 1% by volume TRITON X-100 surfactant.

Pooled human saliva (catalog #IRSHUSML) was obtained from Innovative Research, Novi, MI.

Unless otherwise noted, deionized, 18 megaohm water from a MILLI-Q water purification system (EMD Millipore, Billerica, MA) was used.

### Nonwoven Substrates

Nonwoven substrate A (NWS-A) was a meltblown polypropylene (PP) nonwoven web that was white in color and had an effective fiber diameter of about 12 micrometers, basis weight of about 200 grams per square meter (gsm), solidity of about 10%, and calculated mean pore size of 36 micrometers.

Nonwoven substrate B (NWS-B) was a meltblown polypropylene (PP) nonwoven web that was white in color and had an effective fiber diameter of about 8 micrometers, basis weight of about 200 grams per square meter (gsm), solidity of about 10%, and calculated mean pore size of 24 micrometers.

Nonwoven substrate C (NWS-C) was a meltblown polypropylene (PP) nonwoven web that was white in color and had an effective fiber diameter of about 6 micrometers, basis weight of about 200 grams per square meter (gsm), solidity of about 10%, and calculated mean pore size of 18 micrometers.

Nonwoven substrate D (NWS-D) was a meltblown polypropylene (PP) nonwoven web that was white in color and had an effective fiber diameter of about 4 micrometers, basis weight of about 106 grams per square meter (gsm), solidity of about 10 V, and calculated mean pore size of 12 micrometers.

Nonwoven substrate E (NWS-E) was a meltblown nylon 6.6 nonwoven web that was white in color and had an effective fiber diameter of about 4 micrometers, basis weight of about 130 grams per square meter (gsm), solidity of about 10%, and calculated mean pore size of 13 micrometers.

“Mean pore size” (also known as mean pore diameter) was calculated according to Equation 1.

$$Mean pore size = {d}_{f} ( {( 2 α / π )}^{( - 1 / 2 )} - 1 ) \qquad Equation 1$$

where “d_{f}” is the arithmetic median fiber diameter, and “α” is the web solidity.

### Preparation of Capture Component 2: Nanocellulose Beads Functionalized with SARS-CoV-2 Monoclonal Antibody (mAb)

NANOACT blue colored, nanocellulose beads (catalog #BL BCAW05, carboxylic acid functionalized, 318 nm diameter, 1.05 wt-% suspension in water) were obtained from Asahi Kasei Corporation, Tokyo, Japan. An aliquot of the NANOACT suspension (60 microliters) was added to a pre-weighed 15 mL conical centrifuge tube followed by the sequential addition of 540 microliters of MES (100 millimolar aqueous solution, pH 6), 7.5 microliters of EDC (4 wt.-% aqueous solution), and 15 microliters of sulfo-NHS (4 wt.-% aqueous solution). The tube was maintained at room temperature for 15 minutes and then centrifuged (5000 g) at 20° C. for 20 minutes using a benchtop centrifuge. The resulting supernatant liquid was removed from the tube using a micro-pipette. An aliquot of MES (600 microliters of 100 mM aqueous solution, pH 6) was added to the tube and the suspension was sonicated using a probe sonicator for 10 seconds in pulse mode (set at 3.2 seconds on and 0.5 seconds off). The antibody (60 micrograms of monoclonal antibody (mAb) to SARS-CoV-2 Nucleocapsid, product no. 9547 obtained from Meridian Bioscience, Memphis, TN) was added to the resuspended beads and the tube was vortexed for 5-10 seconds using a Vortex Genie-2 at maximum speed (Scientific Industries. Inc., Bohemia, NY). The suspension was then incubated for 121) minutes at 37° C. in a temperature-controlled chamber. Blocker casein in PBS (7200 microliters) was added and the tube was vortexed for 5-10 seconds and then incubated at 37° C. for (A) minutes. The tube was removed from the temperature-controlled chamber and centrifuged (5000 g) at 20° C. for 20 minutes using a benchtop centrifuge. The resulting supernatant liquid was removed by decanting and then 7200 microliters of boric acid (50 mM aqueous solution, pH 10) was added to the tube. The suspension was sonicated using a probe sonicator for 10 seconds in pulse mode (set at 3.2 seconds on and 0.5 seconds off) followed by centrifuging the tube (5000 g) at 20° C. for 20 minutes. Supernatant was removed by decanting and the resulting conjugated beads were resuspended with a 33 mM aqueous boric acid solution (pH 9.2) that also contained blocker casein (0.2 wt.-%) and sucrose (15 wt.-%). Enough solution was added so that the total weight of the suspension was 1579 mg. This resulted in a 0.038 wt.-% stock suspension of antibody conjugated beads. The suspension was sonicated using a probe sonicator for 10 seconds in pulse mode (set at 3.2 seconds on and 0.5 seconds off). The tubes were stored at 4° C. and used within 2-3 days. Alternatively, the suspension of conjugated beads was aliquoted to 1.5 mL Eppendorf tubes and freeze-dried. The freeze-dried samples were sealed in foil pouches. Each pouch contained two 1 g silica gel desiccant packs (product no. S-3902, obtained from IJLINE Company, Pleasant Prairie, WI) and was stored at room temperature.

### Preparation of Vertical Flow (VF) Detection Devices

Vertical flow detection devices were prepared using different types of nonwoven substrates. Each device consisted of a sealed plastic housing with an internal cavity (external device dimensions: 10 cm (length)×7.5 cm (width)×14 mm (depth) that was prepared by 3D-printing using ACCURA 25 plastic and a 3D Systems PROJET 7000 printer (3D Systems, Rock Hill, SC). The device housing was prepared from two halves (i.e., upper and lower housing sections connected together with latches). The lower housing section of the device contained an internal cavity (dimensions of 51 mm (length)×13 mm (width)×1.5 mm (depth). The upper housing section of the device contained 2 circular openings (each opening 4 mm in diameter). The openings were positioned to be aligned with the cavity section of the lower housing and were spaced apart by 20 mm in the lengthwise direction. The absorbent pads for the device were 50 mm by 13 mm sections cut from WHATMAN Grade GB003 cellulose blotting paper (0.8 mm thick)(obtained from Cytvia, Marlborough. MA). The porous substrate layer of the device was a 50 mm by 13 mm section cut from a single nonwoven substrate selected from Nonwoven Substrates A-E. A stack of three absorbent pads was placed in the cavity of the lower housing and a single section of the selected nonwoven substrate was placed on top of the stack of absorbent pads. The upper and lower housing sections were then mated and secured using magnetic closures to form the finished device. In the finished device, the internal facing surface of the upper housing section pressed against the surface of the nonwoven layer in the stacL The two openings in the upper housing and the nonwoven surface foamed two wells in the device that were used for sample delivery and assay result detection.

### Example 1: Detection of Antigen 1

Two different test samples and a control sample were prepared using human oral swab samples. For each sample, a fresh cheek swab from a human volunteer was obtained using a PURITAN 25-8061 PR Pur-wraps sterile, rayon tipped applicator swab (PURITAN Medical Products, Guilford, ME). Test Sample 1 was prepared by immersing the absorbent material end of a swab into a 1 mL aliquot of buffer solution (50 mM borate buffer (pH 10) that contained NaCl (300 mM) and 1% by volume TRITON X-100 surfactant) that was spiked with Antigen 1 (20 nM). The swab was rotated (10 circular rotations) in the spiked solution by hand rotation of the stick portion of the applicator swab. The spiked sample was maintained for one minute at room temperature and then a 300 microliter aliquot of the spiked sample was added to a microcentrifuge tube that contained 0.00475 mg of Capture Component 2. Capture Component 1 (10 microliters of the 60 micromolar stock solution described above) and MFC (40 microliters of the 0.3% stock solution described above) were mixed together and 25 microliters of the resulting mixture was added to the tube. The sample was maintained at room temperature for 2 minutes before adding to a VF detection device.

Test Sample 2 was prepared using the same procedure as described for Test Sample 1 with the exception that the swab was immersed in a 1 mL aliquot of buffer solution spiked with Antigen 1 (2 nM).

The Control Sample was prepared using the same procedure as described for Test Example 1 with the exception that the swab was immersed in a 1 mL aliquot of buffer solution that was not spiked with Antigen 1.

VF detection devices containing NWS-A were used and a separate device was used for each Test Sample. Either Test Sample 1 or 2 (300 microliters) was added by pipette to the first well opening of the device and the corresponding Control Sample (300 microliters) was added by pipette to the second well opening of the selected device. About 30 seconds after administering the samples, a 100 microliter aliquot of a 0.1M sodium phosphate buffer (pH 8.0) that contained 300 mM NaCl and 1% by volume TRITON X-100 surfactant was added by pipette to each well.

An image of each device was taken using a Canon EOS Rebel T3i digital camera (Canon USA, Melville, NY) in a portable photo booth. The pixel intensity observed in each well of the device was quantified using the Image software program (National Institutes of Heath, Bethesda, MD). The images were convened into 8-bit and inverted prior to quantification. Normalized pixel intensity was obtained for the first well (i.e., sample spiked with Antigen 1) by subtracting the pixel intensity value of the second well (i.e., pixel intensity of the control sample) from the pixel intensity value of the first well. The pixel intensity of the normalized value correlated with the amount of Antigen 1 in the test sample. The results are reported in Table 1.

### Comparative Examples A and B (CE-A and CE-B). Test Samples without MFC Component

Two different comparative test samples and a control sample were prepared according to the procedure of Example 1 with the exception that MFC was not included in the samples. Comparative Test Sample CE-A was prepared by first immersing the swab into a 1 mL aliquot of buffer solution (50 mM borate buffer (pH 10) that contained NaCl (300 mM) and 1% by volume TRITON X-100 surfactant) that was spiked with Antigen 1 (20 nM). The swab was rotated (10 circular rotations) in the spiked solution by hand rotation of the stick portion of the applicator swab. The spiked sample was maintained for one minute at room temperature and then a 300 microliter aliquot of the spiked sample was added to a microcentrifuge tube that contained 0.00473 mg of Capture Component 2. Capture Component 1 (10 microliters of the 60 micromolar stock solution described above) and buffer solution (40 microliters of IX PBS (pH 7.4) that contained 1% by volume TRITON X-100 surfactant) were mixed together and 25 microliters of the resulting mixture was added to the tube. The sample was maintained at room temperature for 2 minutes before adding to a VF detection device.

Comparative Test Sample CE-B was prepared using the same procedure as described for Comparative Test Sample CE-A with the exception that the swab was immersed in a 1 mL aliquot of buffer solution spiked with Antigen 1 (2 nM).

The Control Sample was prepared using the same procedure as described for Comparative Test Sample CE-A with the exception that the swab was immersed in a 1 mL aliquot of buffer solution that was not spiked with Antigen 1. The same procedure as described in Example 1 was used for detecting pixel intensity of a Comparative Test Sample with a VF device. The results are reported in Table 1.

TABLE 1
Normalized Pixel Intensity of first Well
of Device after adding a Test Sample

| | Antigen 1 | | Normalized Pixel |
|---|---|---|---|
| Test | Concentration | MFC Included in | Intensity of |
| Sample | (nM) | Test Sample | Device Well |
| 1 | 20 | Yes | 123.1 |
| 2 | 2 | Yes | 36.4 |
| CE-A | 20 | No | 10.3 |
| CE-B | 2 | No | −0.7 |

### Example 2. Detection of Antigen 1

Pooled human saliva containing 1% by volume TRITON X-100 was dilated 1:1 (volume-volume) with 50 mM borate buffer (pH 10) that contained NaCl 000 mM) and 1% by volume TRITON X-100 surfactant. The diluted saliva was spiked with either 20, 2, 0.5, 0.2, 0.05, or 0 nM Antigen 1. The solution that was not spiked with Antigen 1 (0 nM) was used to prepare the Control Sample. Each solution (300 microliter) was added to a separate microcentrifuge tube that contained 0.00475 mg of Capture Component 2. Capture Component 1 (10 microliters of the 60 micromolar stock solution described above) and MFC (50 microliters of the 0.3% stock solution described above) were mixed together and 30 microliters of the resulting mixture was added to each tube. Each resulting test sample was maintained at room temperature for 5 minutes and the entire volume from a test sample tube was then added by pipette to the first well opening of a VF detection device. Each VF detection device was constructed to contain a single section of NWS-B (polypropylene, EFD=g micrometers) and a single test sample was added to a device. The Control Sample was added by pipette to the second well opening of each device. About 30 seconds after administering the samples, a 100 microliter aliquot of a 0.1M sodium phosphate buffer solution (pH 8.0) that contained 300 mM NaCl and 1% by volume TRITON X-100 surfactant was added by pipette to each well. Images of the devices were taken and analyzed for pixel intensity according to the procedure described in Example 1. The normalized pixel intensity of the fust well of each device (i.e., sample spiked with Antigen 1) is reported in Table 2.

### Comparative Example C (CE-C). Test Samples without Capture Component 1

The same procedure as reported in Example 2 was followed with the exception that the Capture Component 1 of the Test Samples was replaced with a variant that did not have the cellulose binding domain (Capture Component 3). The results are reported in Table 2.

TABLE 2

| | Normalized Pixel Intensity of First Well of | |
|---|---|---|
| Antigen 1 | Device after adding a Test Sample | |
| Concentration | Example 2 | Comparative Example C |
| (nM) | Capture component 1 | Capture component 3 |
| 20 | 133.8 | 20.3 |
| 2 | 88.9 | 8.5 |
| 0.5 | 26.7 | −9.1 |
| 0.2 | 5.9 | −4.9 |
| 0.05 | −5.1 | −5.9 |

### Example 3. Detection of Antigen Using Devices with Different Nonwoven Substrates

The procedure described in Example 1 was followed with multiple test samples prepared by immersing the swab into a 1 mL aliquot of buffer solution (50 mM borate buffer (pH 10) that contained NaCl (300) mM) and 1% by volume TRITON X-100 surfactant) that was spiked with either 20, 2, 0.5, 0.2, or 0.05 nM Antigen 1. In addition. VF devices were modified to contain a single polypropylene nonwoven substrate selected from NWS-A, NWS-B, NWS-C. NWS-D. The normalized pixel intensity of the first well of each device (i.e., sample spiked with SARS-CoV-2 N-Protein) is reported in Table 3.

TABLE 3

| | Normalized Pixel Intensity of First Well of Device | | | |
|---|---|---|---|---|
| | after adding a Test Sample | | | |
| | NWS-A | NWS-B | NWS-C | NWS-D |
| Antigen 1 | Substrate | Substrate | Substrate | Substrate |
| Concentration | (EFD = 12 | (EFD = 8 | (EFD = 6 | (EFD = 4 |
| (nM) | micrometers) | micrometers) | micrometers) | micrometers) |
| 20 | 108.2 | 137.8 | 152.5 | 110.3 |
| 2 | 76.0 | 84.8 | 131.9 | 96.0 |
| 0.5 | 9.7 | 21.1 | 77.8 | Not tested |
| 0.2 | 1.2 | 5.3 | 10.3 | Not tested |
| 0.05 | −5.9 | 4.3 | −10.5 | Not tested |

### Example 4. Detection of Antigen 1 Using a Nylon Substrate

Individual solutions of Antigen 1 (20 nM, 2 nM, 0.5 nM, 0.2 nM, 0.05, and 0 nM) were prepared in 1×PBS (pH 7.4) what contained 1% by volume TRITON X-100 surfactant. The solution that did not contain Antigen 1 (0 nM) was used to prepare the Control Sample. Each solution (300 microliters) was added to a separate microcentrifuge tube that contained 0.00475 mg of Capture Component 2. Capture Component 1 (10 microliters of the 60 micromolar stock solution described above) and MFC (50 microliters of the 0.3% stock solution described above) were mixed together and 30 microliters of the combined solution was added to each tube. Each resulting test sample was maintained at room temperature for 5 minutes and the entire volume from a test sample tube was then added by pipette to the first well opening of a VF detection device. Each VF detection device contained a single section of NWS-E (nylon, EFD=4 micrometers). A single test sample was added to a device. The Control Sample was added by pipette to the second opening of each device. About 30 seconds after administering the samples, a 100 microliter aliquot of a 0.1 M sodium phosphate buffer solution (pH 8.0) that contained 300 mM NaCl and 1% by volume TRITON X-100 surfactant was added by pipette to each well. Images of the devices were taken and analyzed for pixel intensity according to the procedure described in Example 1. The results are reported in Table 4.

TABLE 4

| Analyte 1 | Normalized Pixel Intensity of First Well of |
|---|---|
| Concentration (nM) | Device after adding a Test Sample |
| 20 | 124.1 |
| 2 | 124.1 |
| 0.5 | 43.3 |
| 0.2 | 26.4 |
| 0.05 | −5.3 |

### Comparative Examples D-K (CE-D to CE-K). Comparative Test Samples with Microfibrillated Cellulose (MFC) Replaced with Cellulose Particles

The procedure described in Example 1 was followed with the exception that MFC in the samples was replaced with either 30 microliters of AVICEL PH-101 microcrystalline cellulose stock solution (described above) or 30 microliters of the Sigmacell cellulose stock solution (described above). As used in the examples section, the term solution can include both clear mixtures as well as suspensions (e.g., suspended particles, emulsions, etc.). Each resulting comparative test sample was maintained at room temperature for 5 minutes and the entire volume from a sample tube was then added by pipette to the first well opening of a VF detection device that contained either a single section of NWS-B (polypropylene, EFD=8 micrometers) or NWS-E (nylon, EFD=4). A single comparative test sample was added to a device. The Control Sample was added by pipette to the second well opening of each device. About 30 seconds after administering the samples, a 100 microliter aliquot of a 0.1M sodium phosphate buffer solution that contained 300 mM NaCl and 1% by volume TRITON X-100 surfactant was added by pipette to each well. Images of the devices were taken and analyzed for pixel intensity according to the procedure described in Example 1. The results are reported in Table 5.

TABLE 5

| Compar- | | | | |
|---|---|---|---|---|
| ative | Antigen 1 | Cellulose | Non- | Normalized Pixel |
| Example | Concentra- | Particle Size | woven | Intensity of First |
| (CE) | tion (nM) | (micrometers) | Substrate | Well of Device |
| CE-D | 20 | 50 | NWS-B | −5.4 |
| CE-E | 2 | 50 | NWS-B | −8.4 |
| CE-F | 20 | 50 | NWS-E | −7.3 |
| CE-G | 2 | 50 | NWS-E | −6.6 |
| CE-H | 20 | 20 | NWS-B | −2.4 |
| CE-I | 2 | 20 | NWS-B | −6.0 |
| CE-J | 20 | 20 | NWS-E | −13.2 |
| CE-K | 2 | 20 | NWS-E | −7.4 |

Foreseeable modifications and alterations of this invention will be apparent to those skilled in the art without departing from the scope and spirit of this invention. This invention should not be restricted to the embodiments that are set forth in this application for illustrative purposes. To the extent that there is any conflict or discrepancy between this specification as written and the disclosure in any document mentioned or incorporated by reference herein, this specification as written will prevail.

1. A reagent matrix, comprising:
a plurality of a first capture component comprising a first analyte capture site;
a plurality of a second capture component comprising a second analyte capture site, and at least one of a cellulose binding domain (CBD) or a carbohydrate binding module (CBM), wherein at least one of the first capture component or the second capture components comprises a detection medium; and
a plurality of cellulose nanofibrils.

2. The reagent matrix according to claim 1, wherein the plurality of cellulose nanofibrils comprises a substance selected from the group consisting of fibrillated cellulose, cellulose nanocrystals, and bacterial cellulose.

3. The reagent matrix according to claim 1, wherein the plurality of cellulose nanofibrils has an average surface area of at least 30 m²/g.

4. The reagent matrix according to claim 1, wherein the detection medium comprises a colorimetric material, a fluorescent material, a chemiluminescent material, a phosphorescent material, a radiolabel, a magnetic material, or combinations thereof.

5. The reagent matrix according to claim 1, wherein at least one of the first capture component or the second capture component comprises a particle comprising a substance selected from the group consisting of a synthetic polymer, cellulose, glass, ceramic, and metal.

6. The reagent matrix according to claim 5, wherein the particle comprises cellulose.

7. The reagent matrix according to claim 5, wherein the particles has a particle diameter of at least 0.1 micrometer and at most 10 micrometers.

8. The reagent matrix according to claim 1, wherein the first capture component comprises a protein or a particle.

9. The reagent matrix according to claim 1, wherein the first analyte capture site comprises a substance selected from the group consisting of a monoclonal antibody, a polyclonal antibody, F(ab′) or F(ab′)2 fragments thereof, a capture protein, and an aptamer.

10. The reagent matrix according to claim 1, wherein the second capture component comprises a protein or a particle.

11. The reagent matrix according to claim 1, wherein the second analyte capture site comprises a substance selected from the group consisting of a monoclonal antibody, a polyclonal antibody, F(ab′) or F(ab′)2 fragments thereof, a capture protein, or and an aptamer.

12. The reagent matrix according to claim 1, wherein the second capture component is a bifunctional fusion protein comprising a cellulose binding domain and second analyte capture site.

13. The reagent matrix according to claim 1, wherein the reagent matrix is substantially free of water.

14. The reagent matrix according to claim 1, wherein the reagent matrix further comprises a carrier solution that is an aqueous buffer.

15. A kit for performing an assay for detection of an analyte in a sample, the kit comprising:
a reagent matrix, comprising;
a plurality of a first capture component comprising a first analyte capture site;
a plurality of a second capture component comprising a second analyte capture site, and at least one of a cellulose binding domain (CBD) or a carbohydrate binding module (CBM), wherein at least one of the first capture component or the second capture components comprises a detection medium; and
a plurality of cellulose nanofibrils; and
a porous substrate.

16. The kit according to claim 15, wherein the porous substrate is a nonwoven substrate comprising a substance selected from the group consisting of polyolefin fibers, polyamide fibers, polyester fibers, and fiberglass.

17. The kit according to claim 15, wherein the nonwoven substrate comprises a plurality of microfibers having an effective fiber diameter of at least 0.5 micrometers and at most 16 micrometers.

18. The kit according to claim 15, wherein the porous substrate is a microporous membrane comprising a thermoplastic polymeric material.

19. The kit according to claim 18, wherein the microporous membrane has a mean flow pore size of at least 0.1 micrometers and at most 100 micrometers.

20. The kit according to claim 15, wherein the analyte comprises a substance selected from the group consisting of an antigen, a protein, and a biological contaminant.

21. A method for detection of an analyte in a sample, the method comprising:
combining the sample, a reagent matrix, and a carrier solution in a vessel to form a testing sample, wherein the reagent matrix comprises;
a plurality of a first capture component comprising a first analyte capture site;
a plurality of a second capture component comprising a second analyte capture site, and at least one of a cellulose binding domain (CBD) or a carbohydrate binding module (CBM), wherein at least one of the first capture component or the second capture components comprises a detection medium; and
a plurality of cellulose nanofibrils; and
contacting the testing sample to a porous substrate; and
detecting a signal produced by an aggregation of the analyte with the reagent matrix on a surface of the porous substrate.

22. The method of claim 21, wherein the detecting is by visual inspection.

23. The method of claim 21, wherein the detecting is by an instrument.

24. (canceled)

25. The method according to claim 21, further comprising contacting the reagent matrix with the carrier solution before combining with the sample.

26. The method claim 21, wherein the analyte is selected from the group consisting of an antigen, a protein, and a biological contaminant.
